(19) United States
(12) Patent Application Publication
(10) Pub. No.: US 2024/0191227 A1
(43) Pub. Date: Jun. 13, 2024

(54) Use of CD56 to predict the differentiation potential of muscle stem cells

(71) Applicant: Pensees Inc., Seoul (KR)

(72) Inventor: Jae Ha SHIN, Seoul (KR)

(73) Assignee: Pensees Inc., Seoul (KR)

(21) Appl. No.: 18/147,119

(22) Filed: Dec. 28, 2022

(30) Foreign Application Priority Data
Dec. 8, 2022 (KR) 10-2022-0170914

(51) Int. Cl. C12N 15/11

(52) U.S. Cl. CPC C12N 15/111; C12N 2310/14; C12Q 2600/158

(57) ABSTRACT

The disclosure relates to a composition for detecting a biomarker to predict differentiation potential of a muscle stem cell, including an agent for measuring an mRNA expression level or protein activity level of CD56. Particularly, it is confirmed that among muscle stem cells, cells with high expression or activity levels of CD56 have excellent differentiation potential into myotubes even after continued subculture, and the differentiation potential of muscle stem cells according to the culture period can be predicted in advance, so that it can be usefully used as a tool for effectively evaluating and managing the quality of muscle stem cells.

### CROSS REFERENCE TO RELATED APPLICATION

The present application claims priority to Korean Patent Application No. 10-2022-0170914, filed Dec. 8, 2022, the entire contents of which is incorporated herein for all purposes by this reference.

### BACKGROUND OF THE INVENTION

### Field of the Invention

The disclosure relates to the use of CD56 to predict the differentiation potential of muscle stem cells into myotubes.

### Description of the Related Art

Muscle, the largest organ constituting the human body, undergoes constant disruption and regeneration throughout life, enabling movement in the skeleton, intestines, and heart. The special regenerative ability of muscle is recognized as a key topic in research on maintenance and differentiation mechanisms of adult stem cells, clinical treatment of muscle damage or disease, and prevention of muscle aging.

Muscle stem cells, also called satellite cells (SCs), play an important role in muscle growth and development as a source of cell nuclei during new muscle generation. Muscle stem cells exist between sarcolemma and basal lamina, and are normally inactive, but are activated by stimuli such as trauma to differentiate into new muscles, or to fuse with existing muscle fibers to regenerate muscles. In the muscle regeneration process, quiescence muscle stem cells make myoblasts through cell division, and these myoblasts fuse with damaged muscle fibers or other myoblasts. Some of the activated muscle stem cells do not completely differentiate and form a new muscle stem cell population through self-renewal. It is known that satellite cells maintained in this way account for less than 2% of the cells with nuclei in the entire muscle.

In the in vitro culture of muscle stem cells extracted from muscle, cell yield, doubling time, and differentiation rate also show different characteristics depending on the type of source origin. The characteristics of muscle stem cells, which play an important role in muscle regeneration, determine the muscle growth rate and characteristics of muscles after differentiation. Considering these points, in order to differentiate muscle stem cells into muscle cells with excellent growth rate and characteristics, muscle stem cells with a high differentiation rate should be selected. Therefore, to effectively evaluate and manage the quality of muscle stem cells that can be used as stem cell treatments for various types of degenerative muscular diseases and muscle damage, there is a need to develop a technology for predicting cell lines with excellent differentiation potential into myotubes among isolated muscle stem cells.

### SUMMARY OF THE INVENTION

An object of the disclosure is to provide a composition for detecting a biomarker capable of predicting the differentiation potential of muscle stem cells into myotubes.

Another object of the disclosure is to provide a kit including a composition for detecting a biomarker capable of predicting the differentiation potential of muscle stem cells into myotubes.

Still another object of the disclosure is to provide a method for screening muscle stem cells that have excellent differentiation potential into myotubes and are differentiated into myotubes.

In order to achieve the above object, one aspect of the disclosure provides a composition for detecting a biomarker to predict differentiation potential of a muscle stem cell, including an agent for measuring an mRNA expression level or protein activity level of CD56.

Another aspect of the disclosure provides a kit for predicting the differentiation potential of the muscle stem cell including the composition for detecting the biomarker including an agent for measuring an mRNA expression level or protein activity level of CD56.

Still another aspect of the disclosure provides a method for screening a muscle stem cell to be differentiated into a myotube, including measuring an mRNA expression level or protein activity level of CD56 from an isolated muscle stem cell, and selecting the muscle stem cell whose mRNA expression level or protein activity level of the CD56 is improved compared to a reference value.

The disclosure relates to a composition for detecting a biomarker for predicting the differentiation potential of muscle stem cells, including an agent for measuring the mRNA expression level or protein activity level of CD56. Specifically, since the composition can predict the differentiation potential of muscle stem cells according to the culture period of muscle stem cells in advance by confirming that cells with a high expression or activity level of CD56 among muscle stem cells have excellent differentiation potential into myotubes even if subculture is continued, the composition can be usefully used as a tool to effectively evaluate and manage the quality of the muscle stem cells.

Meanwhile, the effects of the disclosure are not limited to the above-mentioned effects, and other effects could be understood from the following descriptions by a person skilled in the art.

### BRIEF DESCRIPTION OF THE DRAWINGS

FIG. 1 shows the observation of the phenotypes of cell line 1, cell line 2, cell line 3 and cell line 4 among the muscle stem cell lines subcultured until passage 2 (P2), and confirms that there is no difference in the phenotypes of the respective cell lines because the cell shapes are similar.

FIG. 2 shows the rate of cells expressing CD31, a marker for endothelial cells, CD45, a marker for lymphocytes, and CD29 and CD56, markers for muscle cells, among muscle stem cells subcultured until passage 2 (P2) by a flow cytometry, and confirms that unlike cell lines 1 and 2, in cell line 3, CD29 and CD56 were simultaneously expressed at 22.2%, and in cell line 4, CD29 and CD56 were simultaneously expressed at 16.6%, significantly higher than that in cell lines 1 and 2.

FIG. 3 is a photograph taken under a microscope on the 4th day after inducing differentiation from muscle stem cells subcultured until passage 2 (P2) into myotubes, and confirms that when cell line 3 and cell line 4, which had a high rate of cells expressing both CD29 and CD56, were differentiated, the number of myotubes was high, and the differentiation was actively achieved by including a large number of long and thick myotubes.

FIG. 4 shows the expression levels of CD31, CD45, CD29, and CD56 in cell line 1, cell line 2, and cell line 3 at passage 5 (P5), and cell line 3-1 subcultured from cell line 3 to passage 9 (P9) by a flow cytometry, and confirms that in cell line 3, the expression level of CD56 was maintained at P5 as well as at P2.

FIG. 5 shows the observation of cell morphology by taking pictures under a microscope on the 4th day after the start of differentiation to confirm the differentiation potential in cell lines 1, 2, and 3 at passage 5 (P5) and cell line 3 at passage 9 (P9), in which the differentiation into myotubes was induced, and confirms that cell line 3 with a high expression level of CD56 maintained its differentiation potential even after a relatively large number of subcultures.

### DETAILED DESCRIPTION OF THE INVENTION

Hereinafter, terms used in the disclosure are defined.

As used herein, the term “stem cells” refers to cells having not only self-replication ability but also the ability to differentiate into at least two types of cells. Stem cells can be classified into embryonic stem cells, adult stem cells, and induced pluripotent stem cells depending on source origins.

As used herein, the term “muscle stem cell” is a type of adult stem cell and is a pluripotent cell with little cytoplasm found in mature muscle. The muscle stem cells are located between the basement membrane and the sarcolemma that surrounds muscle fibers. Muscle stem cells can differentiate and fuse to strengthen existing muscle fibers and form new ones. Muscle stem cells are involved in the normal growth of muscles as well as regeneration following injuries or diseases. When muscle cells are damaged, quiescent satellite cells are released from beneath the basement membrane and a cell cycle is activated to form new muscle fibers in a process similar to fetal muscle development. After several cell divisions, muscle stem cells begin to fuse with the damaged myotubes and undergo further differentiation and maturation with characteristic peripheral nuclei. As a factor involved in differentiation and proliferation of muscle stem cells, an insulin-induced growth factor IGF-1 is known.

As used herein, the term “differentiation” refers to a phenomenon in which the structure or function of cells is specialized during the division, proliferation, and growth thereof. After differentiating into progenitor cells with a defined lineage, adult stem cells can be further differentiated into other types of progenitor cells and then differentiated into terminally differentiated cells that play a characteristic role in a specific tissue. In a preferred embodiment, the muscle stem cells of the disclosure have the ability to differentiate into myotubes, and the differentiated myotube cells form muscle fibers.

As used herein, the term “myotube” is a cylindrical cell constituting a muscle fiber, which is generated by differentiation of muscle stem cells.

As used herein, the term “CD56 (Cluster of Differentiation 56)” is a type of cell surface antigen cluster for identifying cell surface molecules according to immunophenotype, and is known to be expressed on the surface of natural killer cells (NK cells). In addition, CD 56 is expressed by more immune cells, including alpha beta T cells, gamma delta T cells, dendritic cells, and monocytes. CD56-expressing cell types commonly exhibit potent immunostimulatory effects including T helper 1 cytokine production and efficient cytotoxic capability. Numerous and functional defects of CD56+ immune cells are found in patients with various infectious, autoimmune, or malignant diseases.

As used herein, the term “marker” is a substance that can distinguish a cell line with a high rate of cells differentiated into specific cells from a cell line that does not, and includes organic biomolecules such as proteins, nucleic acids, lipids, glycolipids, glycoproteins, etc. expressed in muscle stem cell lines. As an example of the disclosure is that, when differentiating muscle stem cells into myotubes, a cell line with a high rate of cells expressing CD56 is superior in the differentiation potential into myotubes, compared to a cell line that does not.

Hereinafter, the disclosure will be described in detail.

### 1. Composition for Detecting a Biomarker to Predict the Differentiation Potential of Muscle Stem Cells

One aspect of the disclosure provides a composition for detecting a biomarker for predicting the differentiation potential of muscle stem cells.

The biomarker composition of the disclosure includes an agent for measuring the mRNA expression level or protein activity level of CD56.

The agent for measuring the mRNA expression level of the CD56 may be a single-stranded or double-stranded nucleic acid sequence capable of causing RNA interference with the mRNA of the gene encoding the CD56 protein, and may be a primer pair or probe, an antisense oligonucleotide, a microRNA (miRNA), a small interfering RNA (siRNA), a short hairpin RNA (shRNA), or the like that complementarily binds to the mRNA of the gene encoding the CD56 protein. The single-stranded or double-stranded nucleic acid sequence capable of causing RNA interference with the mRNA of the gene encoding the CD56 protein may consist of a sequence having 70% or more, 75% or more, 80% or more, 85% or more, 90% or more, 95% or more, 99% or more or 100% homology to a part of the mRNA of the gene encoding the CD56 protein. The sequence having the homology to a part of the mRNA of the gene encoding the CD56 protein may have 7 bases or more, 10 bases or more, 13 bases or more, 15 bases or more, 18 bases or more, or 20 bases or more. Meanwhile, a single-stranded or double-stranded nucleic acid sequence capable of causing RNA interference with the mRNA of the gene encoding the CD56 protein may be synthesized chemically or biochemically or in vivo. For example, the single-stranded or double-stranded nucleic acid sequence capable of causing RNA interference with the mRNA of the gene encoding the CD56 protein can be synthesized in vitro by a method such as using RNA polymerase I and administered in vivo, or can be synthesized in vivo by a method such as transcribing antisense RNA using a vector with the origin of the multi-cloning site (MCS) in the opposite direction.

The primer is a nucleic acid sequence with a short free 3′ hydroxyl group, capable of base pairing with a complementary template, and may refer to a short nucleic acid sequence serving as a starting point for copying the template strand.

The probe refers to a natural or modified nucleic acid fragment such as RNA or DNA capable of hybridizing to an mRNA and a specific nucleotide sequence and being labeled so that the presence or absence of a specific mRNA can be identified. The probe used for hybridization may be prepared in the form of an oligonucleotide probe, a single stranded DNA probe, a double stranded DNA probe, an RNA probe, and the like. Conditions suitable for hybridization can be determined by adjusting the temperature, ionic strength (buffer concentration), and the presence of compounds such as organic solvents. These stringent conditions may be determined differently depending on the sequences to be hybridized.

A person skilled in the art can design a primer pair that specifically amplifies a specific region of the gene or a probe that specifically recognizes a specific region of the gene, from the known biomarker gene nucleic acid sequence of the disclosure, and can chemically synthesize the primer pair or probe using methods known in the art. In addition, the primer pair or probe may be modified using a label such as a radioactive isotope, a fluorescent molecule, or biotin to directly or indirectly provide a detectable signal.

The antisense oligonucleotide may refer to DNA or RNA that has a length of 6 to 100 bases, 8 to 60 bases, or 10 to 40 bases and consists of a nucleic acid sequence complementary to the mRNA sequence of the gene encoding the CD56 protein, or a derivative thereof. The antisense oligonucleotide not only inhibits the translation of the mRNA into a protein by binding to the complementary sequence in the mRNA of the gene encoding the CD56 protein, but also inhibits an essential activity for overall biological functions such as translocation of the mRNA of the gene encoding the CD56 protein into the cytoplasm or maturation of the mRNA.

The microRNA may refer to DNA or RNA that has a length of 10 to 40 bases, 12 to 30 bases, or 15 to 25 bases, and consists of a nucleic acid sequence complementary to the 3′-UTR (untranslated region) sequence of the mRNA of the gene encoding the CD56 protein, or a derivative thereof. The microRNA may complementarily bind to the 3′-UTR of the mRNA of the gene encoding the CD56 protein to induce destabilization of the mRNA or inhibit translation of the mRNA.

The small interfering RNA may refer to a double-stranded RNA that has a length of 10 to 40 bases, 12 to 30 bases, or 15 to 25 bases, and consists of an RNA strand of a sense sequence having the same sequence as the mRNA of the gene encoding the CD56 protein, and an RNA strand of an antisense sequence having a sequence complementary thereto. The small interfering RNA complementarily binds to the mRNA of the gene encoding the CD56 protein and induces RNA interference through cleavage of the mRNA, thereby suppressing the expression of the CD56 protein.

The short hairpin RNA may refer to an RNA having a hairpin structure with a loop formed by intramolecular base pairing of a single-stranded RNA in which the sense sequence and antisense sequence of the small interfering RNA for the mRNA of the gene encoding the CD56 protein are linked by 3 to 10 nucleic acid sequences. The short hairpin RNA may be converted into siRNA by a dicer in cells and cause RNA interference.

In addition, the agent for measuring the activity level of the CD56 protein may be a peptide, a peptide mimetics, an aptamer, an antibody, or the like that specifically binds to the CD56 protein.

The peptide that specifically binds to the CD56 protein can be obtained by a commonly used method known in the art, such as phage display.

The peptide mimetics is a peptide or non-peptide that inhibits the binding domain of the CD56 protein, and may be composed of amino acids bound by non-peptide bonds such as psi bonds. In addition, the peptide mimetics is structured similarly to the secondary structural properties of the CD56 protein, can mimic the inhibitory properties of large molecules such as antibodies or water-soluble receptors, and may be a novel small molecule that can act with the same effect as natural antagonists.

The aptamer refers to a single stranded nucleic acid (DNA, RNA or modified nucleic acid) that has a stable tertiary structure, and can bind to the CD 56 protein with high affinity and specificity. The aptamer is comparable to a single antibody due to its unique characteristics of being able to bind to a target molecule with high affinity (usually pM level) and specificity, and has a high potential as an alternative antibody, especially referred to as a ‘chemical antibody’. For the purpose of the disclosure, the aptamer may be used without limitation as long as it binds to the CD56 protein and inhibits its activity.

The antibody may refer to a substance that can specifically bind to the antigenic site of the CD56 protein, and can be prepared by a commonly used method known in the art to which the disclosure pertains. The form of the antibody is not particularly limited, and a polyclonal antibody, a monoclonal antibody, and any part thereof, as long as it has antigen-binding properties, are also included in the antibody of the disclosure, and the antibody of the disclosure may include all immunoglobulin antibodies as well as special antibodies such as humanized antibodies. In addition, the antibody includes not only a complete form having two full-length light chains and two full-length heavy chains, but also a functional fragment of the antibody. The functional fragment of the antibody may refer to a fragment having at least antigen-binding ability, and may be Fab, F(ab′), F(ab′)2, Fv, and the like. For the purpose of the disclosure, the antibody may be used without limitation as long as it is capable of binding to the CD56 protein and inhibiting its activity.

The measurement of mRNA expression or protein activity of CD56 is not limited thereto, but may be preferably selected from the group consisting of a reverse transcriptase polymerase chain reaction, a real-time polymerase chain reaction, a Northern blot, a Western blot, a radioimmunoassay, a radioimmunodiffusion, an immunoprecipitation assay, an immunohistochemical analysis, an enzyme-linked immunosorbent assay (ELISA), a microarray, and a flow cytometry.

The differentiation may be the differentiation of muscle stem cells into myotubes, which are cells constituting muscle fibers.

The differentiation can be preferably performed in a muscle stem cell line subcultured with a number of passages less than 9, more preferably in a muscle stem cell line subcultured with a number of passages less than 7, and even more preferably in a muscle stem cell line subcultured with a number of passages less than 6.

The differentiation from muscle stem cells into muscle-related cells (e.g., myotubes) may include all known methods of differentiating muscle stem cells into muscle-related cells.

The muscle stem cells may be cultured in a commonly used medium containing, for example, Collagenase Type I or Collagenase Type II. More preferably, the muscle stem cells can be cultured in a commonly used medium containing Collagenase Type II.

The muscle stem cells may be cultured in any medium commonly used for culturing stem cells. For example, the muscle stem cells may be cultured in a basic medium such as Dulbecco's Modified Eagle's Medium (DMEM), Minimal Essential Medium (MEM), α-MEM, Basal Medium Eagle (BME), RPMI1640, F-10, F-12, MEM, Glasgow's Minimal Essential Medium (GMEM), Iscove's Modified Dulbecco's Medium (IMDM), etc., more preferably DMEM, but is not limited thereto.

The culture period of the muscle stem cells may be preferably 3 days or more, more preferably 4 days or more, and even more preferably 4 to 30 days.

The muscle stem cells can be cultured in a medium containing 2% horse serum to induce the differentiation of the muscle stem cells into myotubes, and when the culture period exceeds 4 days, the medium is replaced with a fresh medium every 3 to 4 days.

The medium may be supplemented with additives. Generally, the medium may contain a neutral buffer (e.g., phosphate and/or high concentration bicarbonate) in isotonic solution and a protein nutrient (e.g., serum such as FBS, serum replacement, albumin, or essential and non-essential amino acids such as glutamine). Furthermore, it may contain lipids (fatty acids, cholesterol, an HDL or LDL extract of serum) and other ingredients found in most stock media of this kind (e.g., insulin or transferrin, nucleosides or nucleotides, pyruvate, a sugar source such as glucose, selenium in any ionized form or salt, a glucocorticoid such as hydrocortisone and/or a reducing agent such as β-mercaptoethanol).

When the mRNA expression level or protein activity level of CD56 in a specific muscle stem cell line is higher than that in other muscle stem cell lines, it can be predicted that the differentiation potential of the muscle stem cells into myotubes is relatively high.

In addition, when the mRNA expression level or protein activity level of CD56 in a specific muscle stem cell line is higher than a reference value, it can be predicted that the differentiation potential of the muscle stem cells into myotubes is excellent.

Here, a reference value or reference level may refer to a minimum value of the rate of cells expressing CD56 among all muscle stem cells that can be predicted to have excellent differentiation potential of muscle stem cells into myotubes. The minimum value of the rate of cells expressing CD56 among the all muscle stem cells may be 10% to 15%.

According to one embodiment of the disclosure, when the rate of cells expressing CD56 among all muscle stem cells is 10% or more, preferably 13% or more, and more preferably 15% or more, it can be predicted that the differentiation potential of the muscle stem cells into myotube cells is excellent.

Although this reference value or reference level may vary slightly depending on the purpose of differentiation in differentiation into myotubes, it clearly provides an appropriate reference level for classifying cells suitable for differentiation into myotubes.

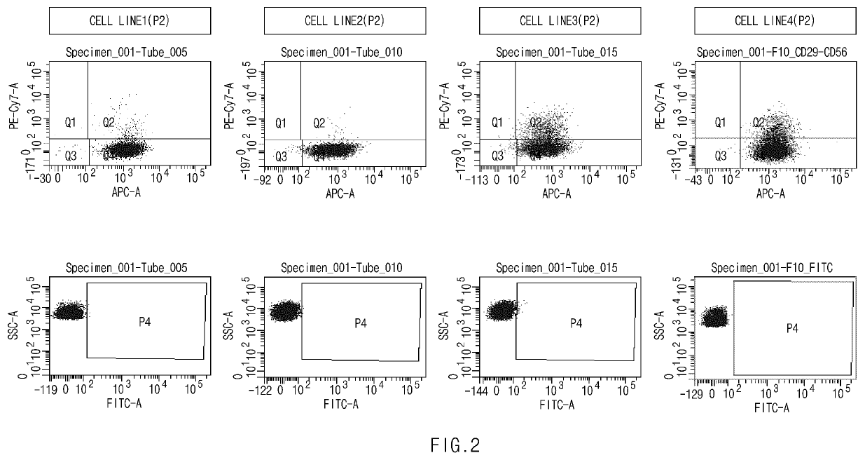

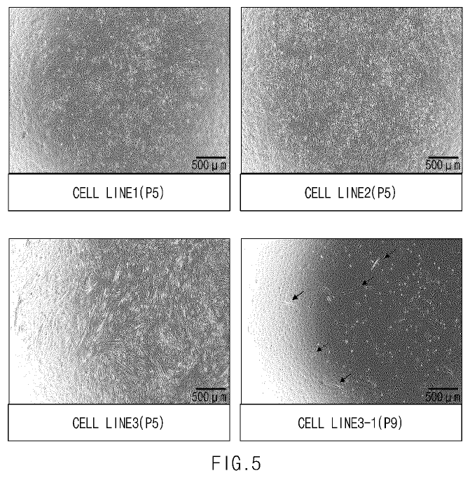

In a specific embodiment of the disclosure, the expression levels of CD31, CD45, CD29, and CD56 in four types of isolated muscle stem cells were confirmed by a flow cytometry, and as a result, it was confirmed that a cell line with a high rate of cells simultaneously expressing CD29 and CD56 was an excellent cell line for differentiation into myotubes (see FIG. 2 and Table 1). In addition, in the case of a cell line with a high rate of cells in which CD29 and CD56 were simultaneously expressed, it was confirmed that the differentiation potential of the cell line was not reduced even after continuous subculture compared to a cell line with a low rate of cells in which CD29 and CD56 were simultaneously expressed, and differentiation into myotubes was well achieved. (see FIG. 5 and Table 2). Furthermore, there are almost no cells expressing CD56 alone in muscle stem cells, so the rate of cells expressing CD29 and CD56 simultaneously can be determined to be the same as the rate of cells expressing CD56 (see Table 1 and Table 2).

Therefore, the biomarker composition including the agent for measuring the expression level or activity level of CD56 of the disclosure can predict the differentiation potential of muscle stem cells according to the culture period in advance, so that the composition can be used as a useful tool for effectively evaluating and managing the quality of muscle stem cells.

### 2. Kit for Predicting the Differentiation Potential of Muscle Stem Cells

Another aspect of the disclosure provides a kit for predicting the differentiation potential of muscle stem cells.

The kit includes an agent for measuring the mRNA expression level or protein activity level of CD56.

The kit may further include an instruction on a method to select muscle stem cells whose mRNA expression level or protein activity level of CD56 is improved compared to a reference value.

The reference value may be a minimum value of the rate of cells expressing CD56 among all muscle stem cells that can be predicted to have excellent differentiation potential of muscle stem cells into myotubes. The minimum value of the rate of cells expressing CD56 among the all muscle stem cells may be 10% to 15%.

According to one embodiment of the disclosure, when the rate of cells expressing CD56 among all muscle stem cells is 10% or more, preferably 13% or more, and more preferably 15% or more, it can be predicted that the differentiation potential of muscle stem cells into myotube cells is excellent.

Although this reference value or reference level may vary slightly depending on the purpose of differentiation in differentiation into myotubes, it clearly provides an appropriate reference level for classifying cells suitable for differentiation into myotubes.

For the description of the agent for measuring the mRNA expression level or protein activity level of CD56 and the differentiation of muscle stem cells, the above description related to “1. Composition for detecting a biomarker to predict the differentiation potential of muscle stem cells” is cited.

The kit includes a primer pair, a probe, a primer pair, a probe antisense oligonucleotide, a microRNA (miRNA), a small interfering RNA (siRNA), or a short hairpin RNA (shRNA) that is specific to the mRNA of the CD56 to measure the expression level of the CD56, or a peptide, a peptide mimetics, an aptamer, and an antibody that specifically binds to the CD56 protein to measure the activity level of the CD56 protein, as well as a tool, a reagent, and the like commonly used in the art used in immunological assays. Examples of such a tool or a reagent include an appropriate carrier, a labeling material capable of generating a detectable signal, a solubilizing agent, a detergent, a buffering agent, a stabilizer, and the like, but are not limited thereto. When the labeling material is an enzyme, the labeling material may include a substrate that can measure the activity of an enzyme and a reaction terminator. The appropriate carrier may be, although not limited thereto, a soluble carrier. e.g., a physiologically acceptable buffer known in the art including PBS, or an insoluble carrier, e.g., polystyrene, polyethylene, polypropylene, polyester, polyacrylonitrile, fluoride resin, cross-linked dextran, polysaccharide, polymer such as magnetic fine particles coated metal on latex, other papers, glass, metal, agarose, and a combination thereof.

The kit may have a form such as a reverse transcriptase polymerase chain reaction (RT-PCR) device, a real-time polymerase chain reaction (Real-time PCR) device, an ELISA plate, a deep-stick device, an immunochromatographic test strip, and a radial partition immunoassay device, and a flow-through device, but is not limited thereto. In addition, when antibodies are provided on a protein chip on which a plurality of proteins can be immobilized, the formation of antigen-antibody complexes for two or more antibodies can be observed.

In a specific embodiment of the disclosure, the expression levels of CD31, CD45, CD29, and CD56 in four types of isolated muscle stem cells were confirmed by a flow cytometry. As a result, it was confirmed that a cell line having a high rate of cells in which CD29 and CD56 were simultaneously expressed was an excellent cell line for differentiation into myotubes (see FIG. 2 and Table 1). In addition, in the case of a cell line with a high rate of cells in which CD29 and CD56 were simultaneously expressed, it was confirmed that differentiation into myotubes was not reduced even after continuous subculture compared to a cell line with a low rate of cells in which CD29 and CD56 were simultaneously expressed, and differentiation into myotubes was well achieved (see FIG. 5 and Table 2). Furthermore, there are almost no cells expressing CD56 alone in muscle stem cells, so the rate of cells expressing CD29 and CD56 simultaneously can be determined to be the same as the rate of cells expressing CD56 (see Table 1 and Table 2).

Therefore, the kit including the agent for measuring the expression level or activity level of CD56 of the disclosure can predict the differentiation potential of muscle stem cells according to the culture period in advance, so the kit can be used as a tool for effectively evaluating and managing the quality of muscle stem cells.

3. Method for Determining Muscle Stem Cells to be Differentiated into Myotubes

Another aspect of the disclosure provides a method for determining muscle stem cells to be differentiated into myotubes.

The method includes measuring the mRNA expression level or protein activity level of CD56 from isolated muscle stem cells, and determining muscle stem cells as the muscle stem cells to be differentiated into myotubes when the mRNA expression level or protein activity level of CD 56 increases compared to a reference value, and the method may be performed in vitro.

For the description of the agent for measuring the mRNA expression level or protein activity level of CD 56 and the differentiation of muscle stem cells, the above description related to “1. Composition for detecting a biomarker to predict the differentiation potential of muscle stem cells” is cited.

The reference value may be a minimum value of the rate of cells expressing CD56 among all muscle stem cells that can be predicted to have excellent differentiation potential of muscle stem cells into myotubes. The minimum value of the rate of cells expressing CD56 among the all muscle stem cells may be 10% to 15%.

According to one embodiment of the disclosure, when the rate of cells expressing CD56 among all muscle stem cells is 10% or more, preferably 13% or more, and more preferably 15% or more, it can be predicted that the differentiation potential of muscle stem cells into myotube cells is excellent.

Although this reference value or reference level may vary slightly depending on the purpose of differentiation in differentiation into myotubes, it clearly provides an appropriate reference level for classifying cells suitable for differentiation into myotubes. The measurement of mRNA expression level or protein activity level is confirmed by the flow cytometry of the rate of cells expressing CD56 expressed on the surface of muscle stem cells among muscle stem cells, so that the rate of cells expressing CD56 was measured in the all muscle stem cells. Accordingly, the degree of differentiation potential into myotubes can be detected according to the rate of cells expressing CD56. As a result, the expression level of CD56 is increased or decreased differently depending on the type of muscle stem cells (i.e., muscle stem cells obtained from different donors or donating), and based on the result, information on whether or not it is suitable muscle stem cell for differentiation into myotubes is provided.

Therefore, in the disclosure, by measuring the differentiation potential of muscle stem cells using CD56, information can be provided to select a muscle stem cell line suitable for differentiation.

Known methods for measuring the expression level of a specific gene or the activity level of a specific protein can be used without limitation as a method for determining the muscle stem cells to be differentiated into myotubes by confirming the expression level of mRNA or the activity level of a protein. Specifically, the degree of expression at the gene level may be measured using a reverse transcriptase polymerase chain reaction (RT-PCR) or a real-time polymerase chain reaction (Real-time PCR), or the degree of activity at the protein level may be measured using a flow cytometry or a Western blotting.

In a specific embodiment of the disclosure, the expression levels of CD31, CD45, CD29, and CD56 in four types of isolated muscle stem cells were confirmed by a flow cytometry. As a result, it was confirmed that the cell line with a high rate of cells in which CD29 and CD56 were simultaneously expressed was an excellent cell line for differentiation into myotubes (see FIG. 2 and Table 1). In addition, in the case of a cell line with a high rate of cells in which CD29 and CD56 were simultaneously expressed, it was confirmed that differentiation into myotubes was not reduced even after continuous subculture compared to a cell line with a low rate of cells in which CD29 and CD56 were simultaneously expressed, and differentiation into myotubes was well achieved (see FIG. 5 and Table 2). Furthermore, there are almost no cells expressing CD56 alone in muscle stem cells, so the rate of cells expressing CD29 and CD56 simultaneously can be determined to be the same as the rate of cells expressing CD56 (see Table 1 and Table 2).

Therefore, the method of determining muscle stem cells to be differentiated into myotubes according to the disclosure can predict the differentiation potential of muscle stem cells according to the culture period in advance, so that the method can be usefully used as a tool for effectively evaluating and managing the quality of muscle stem cells.

### 4. Method for Evaluating the Differentiation Potential of Muscle Stem Cells

Another aspect of the disclosure provides a method for evaluating the differentiation potential of muscle stem cells.

The method may include measuring the mRNA expression level or protein activity level of CD56 from the isolated first and second muscle stem cells and comparing the mRNA expression level or protein activity level of the CD56 of the first and second muscle stem cells, and the method may be performed in vitro.

The method may further include determining the muscle stem cells having the excellent mRNA expression level or protein activity level of the CD56 among the first and second muscle stem cells as muscle stem cells having excellent differentiation potential into myotube cells.

For the description of the agent for measuring the mRNA expression level or protein activity level of the CD56 and the differentiation of muscle stem cells, the above description related to “1. Composition for detecting a biomarker to predict the differentiation potential of muscle stem cells” is cited.

In a specific embodiment of the disclosure, the expression levels of CD31, CD45, CD29, and CD56 in four types of isolated muscle stem cells were confirmed by a flow cytometry. As a result, it was confirmed that the cell line with a high rate of cells in which CD29 and CD56 were simultaneously expressed was an excellent cell line for differentiation into myotubes (see FIG. 2 and Table 1). In addition, in the case of a cell line with a high rate of cells in which CD29 and CD56 were simultaneously expressed, it was confirmed that differentiation into myotubes was not reduced even after continuous subculture compared to a cell line with a low rate of cells in which CD29 and CD56 were simultaneously expressed, and differentiation into myotubes was well achieved (see FIG. 5 and Table 2). Furthermore, there are almost no cells expressing CD56 alone in muscle stem cells, so the rate of cells expressing CD29 and CD56 simultaneously can be determined to be the same as the rate of cells expressing CD56 (see Table 1 and Table 2).

Therefore, the method of evaluating the differentiation potential of muscle stem cells according to the disclosure can predict the differentiation potential of muscle stem cells according to the culture period in advance, so that the method can be usefully used as a tool for effectively evaluating and managing the quality of muscle stem cells.

Hereinafter, the disclosure will be described in detail by examples and experimental examples.

However, the following experimental examples specifically illustrate the disclosure, and the contents of the disclosure are not limited by the following experimental examples.

### Example 1

### Isolation of Muscle Stem Cells

4 g of muscle tissue was extracted from each rump of 4 Korean beef slaughtered on the same day, and it was put in HBSS (Hank's Balanced Salt Solution, Gibco, #14025092) containing 1% AA (Anti-biotic/Anti-myotic, Gibco, #15240-062) to remove blood and sterilize. After mincing the sterilized muscle tissue with a surgical scissor, it was put in a Collagenase Type II solution with a concentration of 1 mg/ml and sufficiently incubated for at least 1 hour while mixing at 37° C. at 80 rpm. The supernatant was collected by centrifugation, and large tissues that were not separated were removed. Impurities were removed through a cell strainer, and the cells were released into a culture medium containing bFGF and cultured in a Petri dish for 1 hour. The supernatant was collected, the non-adherent cells were placed in a culture medium containing amphotericin b, and transferred to a culture dish coated with 1% gelatin.

### Example 2

### Culture and Differentiation of Isolated Muscle Stem Cells

The muscle stem cells (cell line 1, cell line 2, cell line 3, and cell line 4) of the four Korean cattle isolated in Example 1 were cultured in DMEM medium containing 20% serum. Subculture was performed whenever 70 to 80% cell confluency was reached in the culture dish. In order to differentiate the isolated muscle stem cells into myotubes, they were cultured in a culture medium until reaching a cell density of 90% or more in the culture dish. Thereafter, the medium was replaced with DMEM medium containing 2% horse serum, and the medium was exchanged once every 3 to 4 days.

### Example 3

### Observation of the Shapes of Cultured Muscle Stem Cells

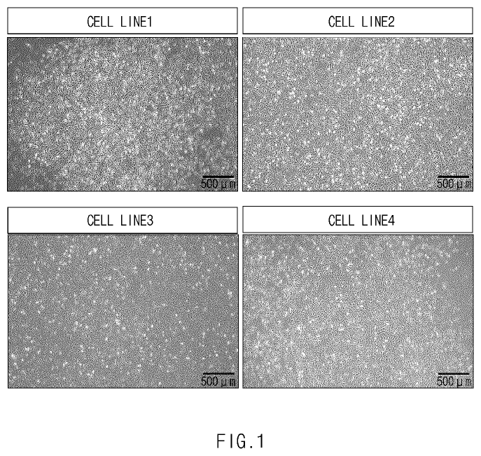

Cell lines 1, 2, 3, and 4 subcultured until passage 2 before differentiation in Example 2 were photographed by phase contrast at 40× magnification with an inverted microscope to determine the phenotype of each muscle stem cell line. As a result, as shown in FIG. 1, cell lines 1, 2, 3, and 4 all had similar cell shapes, and it was confirmed that there was no difference in the phenotypes of the cell lines.

### Example 4

### Analysis of Cell Membrane Markers of Cultured Muscle Stem Cells

The flow cytometry was performed to analyze cell membrane markers for cell lines 1, 2, 3, and 4 subcultured up to passage 2 of Example 3.

Specifically, the expression levels of CD31, a marker for endothelial cells, CD45, a marker for lymphocytes, and CD29 and CD56, markers for muscle cells, were confirmed by a flow cytometry. Each cell line was prepared by suspending in trypsin and washing with PBS. CD31 antibody (Bio-Rad, Cat. No. CD31E1D4), CD45 antibody (Bio-RAD, Cat. No. MCA2220F), CD29 antibody (Biolegend, Cat No. 303008), and CD56 antibody (BD Bioscience, Cat. No. 335826) were added in each cell line and incubated at room temperature for 1 hour. After washing the cells with PBS, the rate of cells expressing each cell membrane marker was measured using a flow cytometer (FACSCanto II, Becton Dickinson).

As a result, as shown in FIG. 2 and Table 1 below, it was confirmed that CD31 and CD45 were hardly expressed in cell line 1, cell line 2, cell line 3 and cell line 4, but CD29, a marker for muscle cells, was expressed at a high rate. In particular, unlike cell lines 1 and 2, cell lines 3 and 4 simultaneously expressed CD29 and CD56 at 22.2% and 16.6%, respectively, which was significantly higher compared to cell lines 1 and 2. In all cell lines, the rate of cells expressing CD56 alone was only 0% or 0.1%, indicating that CD56 was expressed simultaneously with CD29.

TABLE 1

| | Cell | Cell | Cell | Cell |
|---|---|---|---|---|
| | line 1 | line 2 | line 3 | line 4 |
| CD31/CD45-FITC | 0.2 | 0.1 | 0.5 | 0 |
| Negative | 3.2 | 14.9 | 19.3 | 0.2 |
| CD29-APC | 92.6 | 83.1 | 58.4 | 83.2 |
| CD56-PE-Cy7 | 0 | 0 | 0.1 | 0 |
| CD29/CD56(Q2) | 4.2 | 2 | 22.2 | 16.6 |

### Example 5

### Confirmation of the Differentiation Potential of Muscle Stem Cells Induced to be Differentiated

In order to confirm the differentiation potential of the muscle stem cells induced to be differentiated into myotubes in Example 2, the morphology of the cells was observed by taking pictures under a microscope on the 4th day after starting the differentiation.

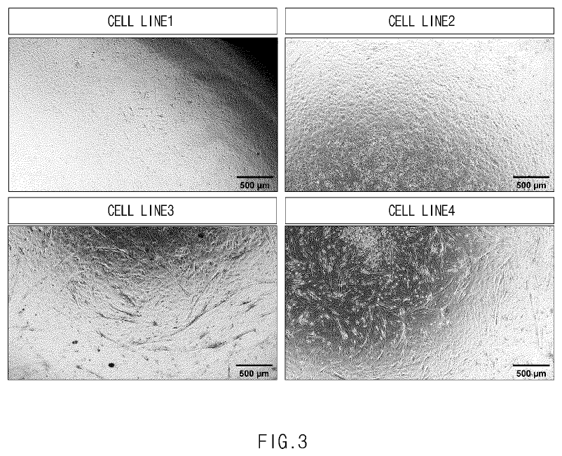

As a result, as shown in FIG. 3, when cell line 3 and cell line 4, which had a high rate of cells expressing CD29 and CD56 simultaneously, were differentiated, it was confirmed that active differentiation was achieved by including a large number of long and thick myotubes. In contrast, when cell line 1 in which the rate of cells expressing CD29 alone was high, but the rate of cells expressing CD29 and CD56 simultaneously was only 4.2%, was differentiated, it was confirmed that differentiation of the cell was induced into about 10 myotubes, but only myotubes with thin thickness and short length were observed. In addition, when cell line 2 in which the rate of cells expressing CD29 alone was high, but the rate of cells expressing CD56 was at 2%, was differentiated, it was confirmed that almost no differentiation into myotubes occurred.

The above results show that the higher the rate of cells simultaneously expressing CD29 and CD56 among the muscle stem cells, the better the differentiation potential into myotubes. In addition, considering that there are almost no cells expressing CD56 alone, the rate of cells expressing CD29 and CD56 simultaneously can be regarded as the rate of cells expressing CD56. Therefore, it can be seen that the higher the rate of cells expressing CD56 among the muscle stem cells, the better the differentiate potential into myotubes.

### Example 6

### Confirmation of Differentiation Potential of Subcultured Muscle Stem Cells

When the in vitro culture of the muscle stem cells continues, the cell characteristics of the muscle stem cells change. In particular, the differentiation potential is greatly reduced through 2 to 3 subcultures. In order to analyze cell membrane markers in cell lines 1, 2, and 3 at passage 5 (P5) and cell line 3 at passage 9 (P9), the flow cytometry was performed in the same manner as in Example 4 above.

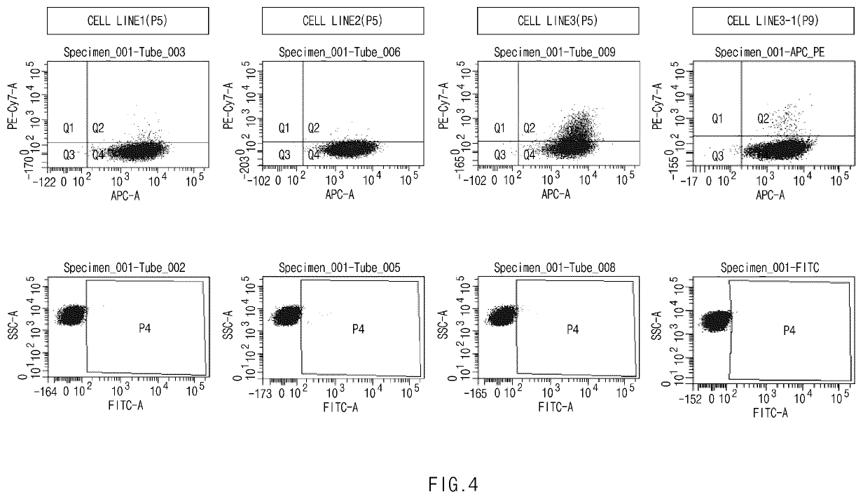

As a result, as shown in FIG. 4 and Table 2 below, CD31 and CD45 were hardly expressed in all cell lines, as in P2, and it was confirmed that endothelial cells and lymphocytes were not included, and in cell lines 1 and 2, CD56 expression was decreased at P5 compared to P2. In contrast, in cell line 3, the expression level of CD56 was maintained at P5 as well as at P2, but significantly decreased at P9.

TABLE 2

| | Cell line | Cell line | Cell line | Cell line |
|---|---|---|---|---|
| | 1(P5) | 2(P5) | 3(P5) | 3(P9) |
| CD31/CD45-FITC | 0.2 | 0.2 | 0.4 | 0.1 |
| Negative | 0.1 | 0 | 0.1 | 1.4 |
| CD29-APC | 98.4 | 99.3 | 75.7 | 95.7 |
| CD56-PE-Cy7 | 0 | 0 | 0 | 0 |
| CD29/CD56(Q2) | 1.5 | 0.7 | 24.1 | 2.9 |

### Example 7

### Confirmation of Differentiation Potential of Subcultured Muscle Stem Cells

In order to confirm the differentiation potential of cell lines 1, 2, and 3 at passage 5 (P5), which were induced to be differentiated into myotubes in Example 6, and cell line 3 at passage 9 (P9), the cells were photographed under a microscope to observe the cell morphology on the 4th day after the start of differentiation.

As a result, as shown in FIG. 5, when cell line 3 at P5, which had a high rate of cells expressing CD29 and CD56 simultaneously, was differentiated, it was confirmed that the number of myotubes was the highest, including many long and thick myotubes, and the most active differentiation was made. However, in the case of P9, which continued subculture, it was confirmed that the number of myotubes was greatly reduced and the differentiation potential was reduced. On the other hand, when cell line 1 and cell line 2 were differentiated, it was confirmed that little differentiation into myotubes occurred.

The above results show that the cell line 3 at P5, which has a high rate of cells expressing CD29 and CD56 simultaneously, maintains its differentiation potential even after a relatively large number of passages. In addition, it is shown that the higher the rate of cells expressing CD56 among muscle stem cells, the better the differentiation potential into myotubes.

Although the disclosure has been described in detail only with respect to the described examples and experimental examples, it is obvious to those skilled in the art that various modifications and variations are possible within the scope of the technical idea of the disclosure, and it is obvious that such variations and modifications fall within the scope of the appended claims.

1.-9. (canceled)

10. A method for screening a muscle-derived stem cell line to be differentiated into a myotube, wherein the stem cell line maintains its differentiation potential after subcultures comprising:
measuring an mRNA expression level or protein activity level of CD56 and CD29 from an isolated muscle-derived stem cell line; and
selecting the muscle-derived stem cell line whose mRNA expression level or protein activity level of CD56 and CD29 is higher compared to a reference value,
wherein the reference value is a minimum value of a rate of a cell expressing CD56 and CD29 among the muscle-derived stem cell line, and the minimum value of the rate of the cell expressing CD56 and CD29 among the muscle-derived stem cell line is 10% to 15%.

11. (canceled)

12. A method for evaluating differentiation maintenance potential of a muscle-derived stem cell line, comprising:
measuring mRNA expression level or protein activity level of CD56 and CD29 from isolated first and second muscle-derived stem cell lines; and
comparing the mRNA expression level or protein activity level of CD56 and CD29 of the first and second muscle-derived stem cell lines.

13. The method of claim 10, wherein the measuring mRNA expression level of CD56 is performed by using one or more selected from the group consisting of a primer pair, a probe, an antisense oligonucleotide, a microRNA (miRNA), a small interfering RNA (siRNA), and a short hairpin RNA (shRNA) that are specific to the mRNA of CD56.

14. The method of claim 10, wherein the measuring mRNA expression level of CD56 is performed by using one or more selected from the group consisting of a peptide, a peptide mimetics, an aptamer, and an antibody that specifically bind to a CD56 protein.

15. The method of claim 10, wherein the measurement is selected from the group consisting of a reverse transcriptase polymerase chain reaction, a real-time polymerase chain reaction, a Northern blot, a Western blot, a radioimmunoassay, a radioimmunodiffusion, an immunoprecipitation assay, an immunohistochemical analysis, an enzyme-linked immunosorbent assay (ELISA), a microarray chip, and a flow cytometry.

16. The method of claim 10, wherein the subcultures are performed with a number of passages less than 9.

17. The method of claim 12, wherein the measuring mRNA expression level of CD56 is performed by using one or more selected from the group consisting of a primer pair, a probe, an antisense oligonucleotide, a microRNA (miRNA), a small interfering RNA (siRNA), and a short hairpin RNA (shRNA) that are specific to the mRNA of CD56.

18. The method of claim 12, wherein the measuring mRNA expression level of CD56 is performed by using one or more selected from the group consisting of a peptide, a peptide mimetics, an aptamer, and an antibody that specifically bind to a CD56 protein.

19. The method of claim 12, wherein the measurement is selected from the group consisting of a reverse transcriptase polymerase chain reaction, a real-time polymerase chain reaction, a Northern blot, a Western blot, a radioimmunoassay, a radioimmunodiffusion, an immunoprecipitation assay, an immunohistochemical analysis, an enzyme-linked immunosorbent assay (ELISA), a microarray chip, and a flow cytometry.

20. The method of claim 12, wherein the subcultures are performed with a number of passages less than 9.

21. The method of claim 12, wherein the method further comprises determining the muscle-derived stem cell line having higher mRNA expression level or protein activity level of the CD56 and CD29 among the first and second muscle-derived stem cell cells as muscle-derived stem cells having higher differentiation maintenance potential into myotube cells.
